(19) United States
(12) Patent Application Publication
(10) Pub. No.: US 2023/0106523 A1
(43) Pub. Date: Apr. 6, 2023

(54) QUERCETIN ENHANCEMENT FORMULATION

(71) Applicant: SMARTDNA PTY LTD, Clayton (AU)

(72) Inventors: Margaret SMITH, Clayton, Victoria (AU); Simone WALSH, Clayton, Victoria (AU); Grant WASHINGTON-SMITH, Clayton, Victoria (AU)

(21) Appl. No.: 17/907,239

(22) PCT Filed: Mar. 25, 2021
(86) PCT No.: PCT/AU2021/050267

§ 371 Date: Sep. 23, 2022

(30) Foreign Application Priority Data
Mar. 26, 2020 (NZ) 762991
Apr. 16, 2020 (AU) 2020901205

(51) Int. Cl. A61K 31/37; A61K 36/752; A61K 33/30; A61K 31/375; A61P 43/00

(52) U.S. Cl. CPC A61K 31/37; A61K 36/752; A61K 33/30; A61K 31/375; A61P 43/00

(57) ABSTRACT

Compositions comprising 6,7-dihydroxybergamottin (6,7-DHB) and quercetin wherein the bioavailability of quercetin is enhanced as a result of the inhibition of cytochrome P450 enzymes from 6,7-DHB. The compositions are also used in methods of treating cancer, inflammation, cardiovascular disease, hypertension, polycystic ovary syndrome, a neurodegenerative disease, an infection or for enhancing cellular zinc uptake.

### 1. FIELD OF THE INVENTION

The invention relates to compositions and their use in promoting health and treating conditions. More particularly, the invention relates to a composition comprising quercetin, and uses thereof.

### 2. BACKGROUND

Quercetin (2-(3,4-dihydroxyphenyl)-3,5,7-trihydroxy-4H-chromen-4-one) is a flavonoid widely distributed in nature and produced naturally by several plants. It can be obtained from a number of sources including many fruits, vegetables, leaves, seeds, and grains; red onions and kale are particularly high in quercetin. Quercetin has been reported to inhibit the oxidation of other molecules and hence is classified as an antioxidant. (Williams R J, Spencer J P, Rice-Evans C (April 2004), Russo G L, Russo M, Spagnuolo C, Tedesco I, Bilotto S, lannitti R, Palumbo R (2014).

As a result of extensive media coverage of these effects, quercetin supplements have become increasingly popular. However quercetin is thought to be rapidly conjugated to form glucuronide or sulphate derivatives, both of which are relatively inactive. Some human studies have indicated that the bioavailability of active quercetin is no more than 10 minutes (Graefe E U, Derendorf H, Veit M (1999). Therefore, there is a need for a mechanism by which quercetin bioavailability can be increased or to at least provide the public with a useful choice.

### 3. SUMMARY OF THE INVENTION

The inventors have found that co-administration of quercetin and 6′,7′-dihydroxybergamottin (6,7-DHB) increases the bioavailability of quercetin in human and animal subjects. Furthermore, the inventors have produced compositions comprising quercetin and 6,7-DHB, which can administered to a subject to increase the bioavailability of quercetin in the subject, thereby enhancing and/or prolonging its biological activity.

In one aspect, the present invention provides a composition comprising quercetin and 6,7-dihydroxybergamottin (6,7-DHB), wherein 6,7-DHB is present in an amount sufficient to enhance the bioavailability of quercetin.

In conventional quercetin supplements, the typical amount of quercetin can be as high as 5 g, due to its poor bioavailability. The compositions of the present invention comprise 6,7-DHB, which increases bioavailability of quercetin and thereby advantageously reduces the amount of quercetin required in the composition. Thus, in one embodiment, the quercetin is present in an amount of less than 5 g, less than 3 g, less than 2 g, or less than 1 g.

In one embodiment, the quercetin is present in an amount in the range of about 50 mg to about 1,500 mg. In one embodiment, the quercetin is present in an amount in the range of about 100 mg to about 1,500 mg, or about 250 mg to about 750 mg, or about 400 mg to about 600 mg.

In one embodiment, the quercetin is present in an amount in the range of about 400 mg to about 600 mg. In one embodiment, the quercetin is present in an amount in the range of about 500 mg to about 550 mg. In one embodiment, the quercetin is present in an amount of about 526 mg.

In another embodiment, the quercetin is present in an amount in the range of about 50 mg to 100 mg.

In one embodiment, the 6,7-DHB is present in an amount in the range of about 1 mg to about 50 mg, or about 2 mg to about 20 mg, or about 4 mg to about 10 mg.

In one embodiment, the 6,7-DHB is present in an amount of about 4 mg to about 10 mg. In one embodiment, the 6,7-DHB is present in an amount of about 5 mg, or about 6 mg, or about 7 mg or about 8 mg. In one embodiment, the 6,7-DHB is present in an amount of about 6.75 mg.

The 6,7-DHB can be in a purified, partially purified, or in a natural form. Preferred natural sources of 6,7-DHB include Urtica dioica extract (nettle root extract), pomelo extract (Citrus maxima extract), pummelo extract (Citrus grandis extract) or NZ grapefruit extract (NZGF extract).

In one embodiment, the 6,7-DHB is present in the form of a nettle root extract or a grapefruit extract. The amounts of 6,7-DHB recited herein refer to the amount of actual 6,7-DHB present, not the total amount of extract (which contains additional molecules other than 6,7-DHB).

In one embodiment, the nettle root extract or grapefruit extract comprises 6,7-DHB in an amount of at least 20%, at least 25%, at least 30%, at least 35%, at least 40%, or at least 45% by weight.

In one embodiment, the composition comprises
(a) quercetin in an amount in the range of about 400 mg to about 600 mg; and
(b) 6,7-DHB in an amount in the range of about 4 mg to about 10 mg.

In one embodiment, the composition comprises one or more or all of the following:
(a) zinc;
(b) vitamin D3;
(c) vitamin C;
(d) Piper nigrum fruit extract; and
(e) ginger root extract.

In one embodiment, the composition comprises zinc. In one embodiment, the zinc is present in an amount of about 1 mg to about 100 mg, about 1 mg to about 50 mg, about 1 mg to about 10 mg, or about 1 mg to about 5 mg. In one embodiment, the zinc is present in an amount of about 3.3 mg.

Suitable forms (e.g., salts) of zinc will be known by those skilled in the art. In one embodiment, the zinc is present in the form of zinc gluconate. In one embodiment, the zinc gluconate is present in an amount of about 1 mg to about 100 mg, about 5 mg to about 50 mg, or about 10 mg to about 30 mg. In one embodiment, the zinc gluconate is present in an amount of about 23 mg. As a person skilled in the art will appreciate, 23 mg of zinc gluconate contains about 3.3 mg of zinc.

In one embodiment, the composition comprises vitamin D. In one embodiment, the vitamin D is vitamin D3. In one embodiment, the vitamin D is present in an amount of about 1 mg to about 50 mg, about 1 mg to about 20 mg, or about 2 mg to about 10 mg. In one embodiment, the vitamin D is present in an amount of about 5 mg.

In one embodiment, the composition comprises vitamin C. In one embodiment, the vitamin C is present in an amount of about 5 mg to about 200 mg, about 10 mg to about 100 mg, or about 30 mg to about 70 mg. In one embodiment, the vitamin C is present in an amount of about 55 mg.

In one embodiment, the composition comprises Piper nigrum fruit extract. In one embodiment, the Piper nigrum fruit extract is present in an amount of about 1 mg to about 50 mg, about 1 mg to about 20 mg, or about 2 mg to about 10 mg. In one embodiment, the Piper nigrum fruit extract is present in an amount of about 5 mg.

In one embodiment, the composition comprises ginger extract. In one embodiment, the ginger extract is present in an amount of about 5 mg to about 100 mg, about 10 mg to about 80 mg, or about 30 mg to about 50 mg. In one embodiment, the ginger extract is present in an amount of about 40 mg.

In one embodiment, the composition comprises one or more nutraceutically acceptable excipients. In one embodiment, the nutraceutically acceptable excipients include one or two or all of microcrystalline cellulose, colloidal silica, and magnesium stearate.

In one embodiment, the composition comprises D-ribose-L-cysteine. In one embodiment, the D-ribose-L-cysteine is present in an amount in the range of about 25 mg to about 500 mg. In one embodiment, the D-ribose-L-cysteine is present in an amount in the range of about 75 mg to about 250 mg. In one embodiment, the D-ribose-L-cysteine is present in an amount of about 125 mg.

The composition may be in any suitable form for administration to a subject. For example, the composition may be dry or liquid. In one embodiment, the composition is a dry composition. In one embodiment, the composition is suitable for oral administration. In one embodiment, the composition is a capsule or tablet.

In one embodiment, the composition comprises nanoparticles (e.g., chitosan nanoparticles), zein, poly(lactic-co-glycolic acid) (i.e., PGLA), or polymeric micelles.

In one embodiment, the composition is a nutraceutical composition. In one embodiment, the composition is a pharmaceutical composition.

In one embodiment, the composition is an animal feed. In one embodiment, the composition is a fish feed.

In one embodiment, the total mass of the composition is less than 2 g, less than 1 g, less than 800 mg, less than 700 mg, or less than 600 mg.

In one embodiment, the composition comprises one or more additional active agents. In one embodiment, the one or more additional active agents are selected from the group consisting of vitamins, antioxidants, coenzyme Q-10, genistein, resveratrol, curcumin, lycopene, polyunsaturated fatty acids including omega-3 fatty acid and polyphenolics. In one embodiment, the composition comprises one or more fat soluble vitamins.

In another aspect, the present invention provides a composition comprising:
(a) quercetin, in an amount in the range of about 400 mg to about 600 mg;
(b) 6,7-DHB, in an amount in the range of about 4 mg to about 10 mg;
(c) zinc, in an amount in the range of about 1 mg to about 10 mg;
(d) vitamin D3, in an amount in the range of about 2 mg to about 10 mg;
(e) Piper nigrum fruit, in an amount in the range of about 2 mg to about 10 mg; and
(f) ginger extract, in an amount in the range of about 30 mg to about 50 mg.

In another aspect, the invention provides a nutraceutical composition comprising quercetin and a source of 6′,7′-Dihydroxybergamottin (6,7-DHB), which includes Urtica dioica extract (nettle root extract), pomelo extract (Citrus maxima extract), pummelo extract (Citrus grandis extract) or NZ grapefruit extract (NZGF extract), and optionally nutraceutically acceptable excipients.

In another aspect, the invention provides a nutraceutical composition consisting essentially of quercetin and 6,7-DHB.

In one embodiment the nutraceutical composition is a food or beverage, or supplement for food or beverage. In one embodiment, the 6,7-DHB is powdered extract. In one embodiment, the 6,7-DHB is from NZGF juice or nettle root.

In another aspect, the present invention provides a method of preventing or treating a condition in a subject, which condition would benefit from administration of quercetin, the method comprising administering the composition of the invention to the subject.

In another aspect, the present invention provides a method of preventing or treating a condition in a subject, which condition would benefit from administration of quercetin, the method comprising administering to the subject 6,7-DHB, simultaneously or sequentially with quercetin.

The condition can be any disease, injury or other ailment which may benefit from quercetin's antioxidant and/or ionophore activity. In one embodiment, the condition would benefit from administration of quercetin is a cancer, inflammation, cardiovascular disease, hypertension, polycystic ovary syndrome, a neurodegenerative disease, or an infection.

In one embodiment, the condition is Alzheimer's disease, motor neuron disease, epilepsy, multiple sclerosis or Parkinson's disease.

In one embodiment, the condition is glutamate toxicity.

In one embodiment, the condition is a heart disease or stroke.

In one embodiment, the condition is osteoporosis, arthritis, or rheumatoid arthritis.

In one embodiment, the condition is hypertension, asthma, chronic obstructive pulmonary disease (COPD), pneumonia, chronic bronchitis or lung cancer.

In one embodiment, the condition is diabetes or cancer. In one embodiment, the cancer is lymphoma or leukemia.

In one embodiment, the condition is cancer, inflammation, ageing or oxidative stress.

In one embodiment, the condition is a viral infection or a bacterial infection. In one embodiment, the viral infection is a hepatitis C virus infection. As described herein, quercetin acts as an ionophore, thereby enhancing cellular uptake of zinc, which has antiviral properties (see e.g., Read et al., 2019).

In one embodiment, the condition is polycystic ovary syndrome.

In one embodiment, the condition is a wound.

In one embodiment, the condition is an allergy, asthma, hay fever, or hives.

In one embodiment, the condition is an inflammatory disorder. In one embodiment, the condition is neuroinflammation or psoriasis.

In one embodiment, the condition is inflammatory bowel disease, Crohn's disease, or irritable bowel syndrome.

In one embodiment, the condition is a mental disorder. In one embodiment, the condition is depression or anxiety.

In one embodiment, the condition is histamine intolerance.

In one embodiment, the condition is alopecia or alopecia areata.

In another aspect, the invention provides a method of increasing the bioavailability of quercetin in a subject, the method comprising administering to the subject 6,7-DHB simultaneously or sequentially with quercetin.

In another aspect the invention provides a method of preventing or treating cancer in a subject, the method comprising administering to the subject 6,7-DHB simultaneously or sequentially with quercetin. In some embodiments, the quercetin is administered simultaneously or sequentially with an anti-cancer therapy, such as a chemotherapeutic agent.

In another aspect the invention provides a method of preventing or treating inflammation in a subject, the method comprising administering to the subject 6,7-DHB simultaneously or sequentially with quercetin.

In another aspect the invention provides a method of enhancing cellular zinc uptake in a subject, the method comprising administering to the subject 6,7-DHB simultaneously or sequentially with quercetin.

In another aspect the invention provides a method of enhancing probiotic activity in a subject, the method comprising administering to the subject 6,7-DHB simultaneously or sequentially with quercetin.

In another aspect the invention provides the use of a 6,7-DHB in the manufacture of a medicament for increasing the bioavailability of quercetin in a subject, wherein the medicament is to be administered simultaneously or sequentially with quercetin.

In another aspect the invention provides a use of 6,7-DHB in the manufacture of a medicament for preventing or treating cancer in a subject, wherein the medicament is to be administered simultaneously or sequentially with quercetin.

In another aspect the invention provides a use of 6,7-DHB in the manufacture of a medicament for preventing or treating inflammation in a subject, wherein the medicament is to be administered simultaneously or sequentially with quercetin.

In another aspect the invention provides the use of 6,7-DHB and quercetin in the manufacture of a medicament for preventing or treating cancer in a subject.

In another aspect the invention provides the use of 6,7-DHB and quercetin in the manufacture of a medicament for preventing or treating cardiovascular disease in a subject.

In another aspect the invention provides the use of 6,7-DHB and quercetin in the manufacture of a medicament for preventing or treating dysbiosis of the gut microbiome in a subject.

In another aspect the invention provides the use of 6,7-DHB and quercetin in the manufacture of a medicament for preventing or treating inflammation in a subject.

In another embodiment the invention provides a nutraceutical composition comprising 6,7-DHB and quercetin for use in increasing the bioavailability of quercetin in a subject.

In another embodiment the invention provides a nutraceutical composition comprising 6,7-DHB and quercetin for use in preventing or treating cancer in a subject.

In another embodiment the invention provides a nutraceutical composition comprising 6,7-DHB and quercetin for use in preventing or treating inflammation in a subject.

In another aspect the invention provides a nutraceutical kit for increasing the bioavailability of quercetin, the kit comprising (a) quercetin and (b) 6,7-DHB, wherein the components of the kit are to be administered simultaneously or sequentially.

In one embodiment, the 6,7-DHB is administered simultaneously or sequentially with one or more additional active agents. In one embodiment, the one or more additional active agents are selected from the group comprising vitamins, antioxidants, coenzyme Q-10, genistein, resveratrol, curcumin, lycopene, polyunsaturated fatty acids including omega-3 fatty acid and polyphenolics.

In one embodiment, the 6,7-DHB is to be administered simultaneously or prior to administration of quercetin.

In one embodiment, the 6,7-DHB and quercetin are administered in the same composition. In one embodiment, the composition is the composition of the invention.

In one embodiment, the 6,7-DHB and quercetin are administered in separate compositions.

In one embodiment, the 6,7-DHB is administered to the subject prior to quercetin.

In one embodiment, the 6,7-DHB is administered about 0.5 to about 12 hours prior to quercetin. In one embodiment, the 6,7-DHB is administered about 0.5 to about 6 hours prior to quercetin.

In one embodiment, the 6,7-DHB is administered about 1 to about 4 hours prior to quercetin. In one embodiment, the 6,7-DHB is administered about 2 hours prior to quercetin.

In one embodiment, quercetin is administered in an amount in the range of about 100 mg to about 1,500 mg, or about 250 mg to about 750 mg, or about 400 mg to about 600 mg per dose.

In one embodiment, quercetin is administered in an amount in the range of about 400 mg to about 600 mg per dose. In one embodiment, the quercetin is administered in an amount in the range of about 500 mg to about 550 mg per dose. In one embodiment, the quercetin is administered in an amount of about 526 mg per dose.

In one embodiment, 6,7-DHB is administered in an amount in the range of about 1 mg to about 50 mg, or about 2 mg to about 20 mg, or about 4 mg to about 10 mg per dose.

In one embodiment, 6,7-DHB is administered in an amount of about 4 mg to about 10 mg per dose. In one embodiment, the 6,7-DHB is administered in an amount of about 5 mg, or about 6 mg, or about 7 mg or about 8 mg per dose. In one embodiment, the 6,7-DHB is administered in an amount of about 6.75 mg per dose.

In one embodiment,
(a) quercetin is administered in an amount in the range of about 400 mg to about 600 mg per dose; and
(b) 6,7-DHB is administered in an amount in the range of about 4 mg to about 10 mg.

In one embodiment, one or two doses of quercetin are administered per day and/or one or two doses of 6,7-DHB are administered per day.

In one embodiment, the methods further comprise administering zinc to the subject. As described herein, quercetin is a zinc ionophore and thus enhances the cellular uptake of zinc.

In one embodiment, zinc is administered in an amount in the range of about 1 mg to about 20 mg per dose. In one embodiment, zinc is administered in an amount in the range of about 1 mg to about 5 mg per dose. In one embodiment, the zinc is administered in an amount of about 3.3 mg per dose.

In one embodiment, the subject is an animal. In one embodiment, the subject is a mammal. In one embodiment, the subject is a human.

This compositions described herein can also be used as a feed for animals, including fish. Thus, in one embodiment, the subject is a fish.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### 4. FIGURES

Embodiments of the invention will now be described with reference to the drawings in which:

FIG. 1 is a graph showing blood serum levels of quercetin following ingestion of (a) quercetin (80 mg+200 ml filtered water) and (b) 6,7 DHB followed by quercetin (80 mg quercetin and 200 ml grapefruit extract) in a human subject.

FIG. 2 is a graph showing blood serum levels of quercetin following ingestion of (a) quercetin and (b) 6′,7′ DHB and quercetin in rats.

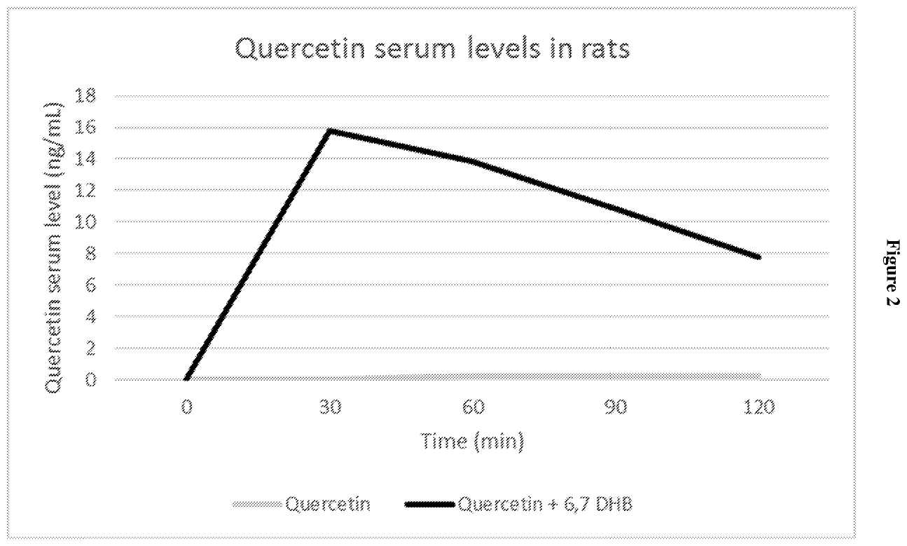

FIG. 3 is a graph showing blood serum levels of quercetin following ingestion of (a) quercetin and Ruby Red grapefruit and (b) quercetin and NZ grapefruit (6′,7′ DHB) in rats.

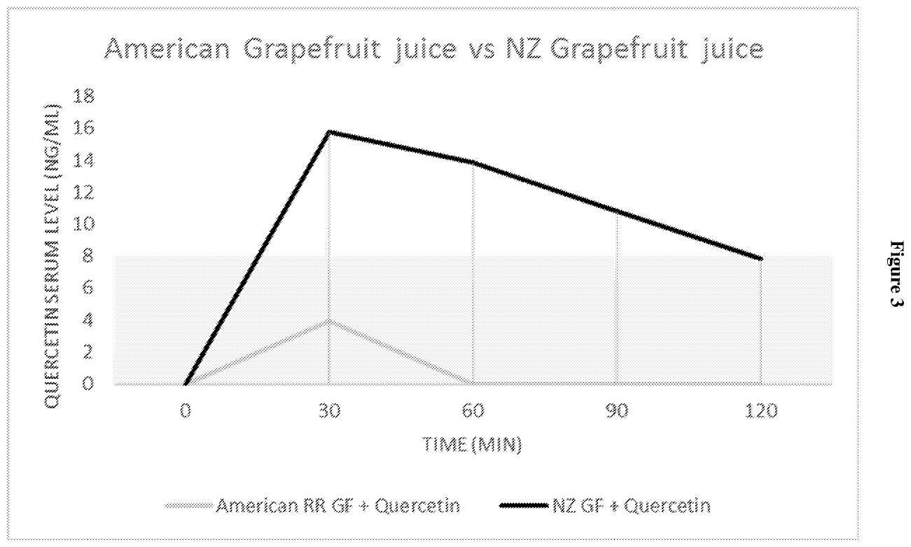

### 5. DETAILED DESCRIPTION OF THE INVENTION

### 5.1 Definitions

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner

The terms “New Zealand grapefruit extract” as used in this specification mean any substance obtained from a New Zealand grapefruit including the juice, flesh, pulp, pith, peel, seeds and mixtures thereof.

The term “bioavailability” as used herein with reference to quercetin, refers to the concentration of free quercetin present in the blood serum of a subject to whom quercetin has been administered. An increase in bioavailability may be indicated by a higher concentration of free quercetin in the blood serum at a given time following administration, and/or by the presence of free quercetin in the blood serum for a longer time period following administration, relative to when quercetin is administered by itself.

The term “New Zealand grapefruit” (NZGF) as used here means a Citrus paradise hybrid that is known in New Zealand as a New Zealand grapefruit, Poor Man's orange or Goldfruit. New Zealand grapefruit is a generally seedy, yellow/orange-fleshed fruit whose trees can be characterized by a bark condition resembling psoriasis. While the fruit and tree are similar to true grapefruit, the New Zealand grapefruit is thought to be a pomelo hybrid or tangelo that originated in China or Australia. New Zealand grapefruit reach maturity early compared to other grapefruit varieties and have a much lower heat requirement, allowing them to be grown in temperature regions such as New Zealand which are too cold for true grapefruit.

“Quercetin” is a plant flavonol from the flavonoid group of polyphenols. It is found in many fruits, vegetables, leaves, seeds, and grains; red onions and kale are common foods containing appreciable amounts of quercetin. Quercetin is a flavonoid widely distributed in nature. The name has been used since 1857, and is derived from quercetum (oak forest), after Quercus. It is a naturally occurring polar auxin transport inhibitor. The bioavailability of quercetin in humans is low and highly variable (0-50%), and it is rapidly cleared with an elimination half-life of 1-2 hours after ingesting quercetin foods or supplements. Following dietary ingestion, quercetin undergoes rapid and extensive metabolism that makes the biological effects presumed from in vitro studies unlikely to apply in vivo. Quercetin is one of the most abundant dietary flavonoids, with an average daily consumption of 25-50 milligrams Quercetin has the following chemical structure:

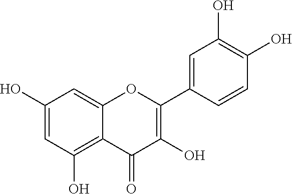 [SMILES: O=c1c(O)c(-c2ccc(O)c(O)c2)oc2cc(O)cc(O)c12]

6,7-Dihydroxybergamottin, or “6,7-DHB”, is a natural furanocoumarin found in pomelos, grapefruits, and sour oranges, in both the peel and the pulp, as well as nettle root and other sources. Along with the chemically related compound bergamottin, it is believed to be responsible for a number of grapefruit—drug interactions. 6,7-DHB has the following chemical structure:

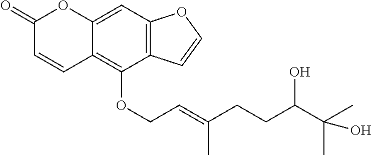 [SMILES: C/C(=C\COc1c2ccoc2cc2oc(=O)ccc12)CCC(O)C(C)(C)O]

The term “treating” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration of decline; making the final point of degeneration less debilitating; improving the patient's physical or mental well-being.

As used herein, the terms “preventing”, “prevent” or “prevention” refer to administration of a therapy (e.g., the composition of the present invention) to stop or hinder the development of at least one symptom of a condition.

As used herein, the term “condition” refers to a disruption of or interference with normal function, and is not to be limited to any specific condition, and will include diseases, disorders, injuries, or other ailments.

The term “subject” as used herein is preferably human but includes animals. Preferred “animals” are companion animals and farm animals Examples of companion animals are dogs, cats, birds, aquarium fish, guinea pigs, (jack) rabbits, hares and ferrets. Examples of farm animals are aquaculture fish, pigs, horses, ruminants (cattle, sheep and goats) and poultry.

The term “cancer” as used herein refers to all types of cancer, neoplasm, or malignant tumors found in mammals, including leukemia, carcinomas and sarcomas. Exemplary cancers include cancer of the brain, breast, cervical, colon, head & neck, liver, kidney, lung, non-small cell lung, melanoma, mesothelioma, ovary, sarcoma, stomach, uterus and Medulloblastoma. Additional examples include, Hodgkin's Diseases, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “cardiovascular disease” as used herein refers to a class of diseases that involve the heart or blood vessels. Cardiovascular disease includes coronary artery diseases (CAD) such as angina and myocardial infarction (commonly known as a heart attack). Other cardiovascular diseases include stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis.

The term “Dysbiosis” as used herein refers to a term for a microbial imbalance or maladaptation on or inside the body, such as an impaired microbiota. For example, a part of the human microbiota, such as the gut flora, or vaginal flora, can become dysbiotic, with normally dominating species underrepresented and normally outcompeted or contained species increasing to fill the void

The term “nutraceutical” as used herein denotes usefulness in both nutritional and pharmaceutical fields of application. Thus, novel nutraceutical compositions can be used as supplements to food and beverages, dietary supplements and as pharmaceutical formulations for enteral or parenteral application which may be solid formulations, such as capsules or tablets, or liquid formulations, such as solutions or suspensions.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, preferably +/−10%, or more preferably +/−5%, of the designated value.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### 5.2 Compositions and Methods of the Invention

The inventors have now found that 6,7-DHB has a surprisingly efficacious effect on the bioavailability of quercetin.

Increasing the bioavailability of quercetin increases the extent and period of its biological activity, leading to positive health results.

Without being bound by theory, it is believed that the increased bioavailability of quercetin observed by the present inventors is due to 6,7-DHB inhibiting the enzyme isoform CYP3A4, a member of the P450 superfamily of enzymes.

CYP3A4 is predominantly found in the liver, but is also present in the intestine, where it plays an important role in the metabolism of certain drugs. In particular, CYP3A4 catalyses reactions of prodrugs, which convert these into the active form necessary for activity.

For this reason, people taking pharmaceutical drugs are advised not to take grapefruit, as it may interfere with the drug including increasing its potency.

In one embodiment, the composition of the invention is a nutraceutical composition. In one embodiment the nutraceutical composition of the invention consists essentially of quercetin and 6,7-DHB. In another embodiment the nutraceutical composition comprises quercetin, 6,7-DHB and optionally nutraceutically acceptable excipients.

The 6,7-DHB may be in the form of a liquid such as the juice of NZGF, or a blend of NZGF components including one or more of the juice, flesh, pulp, pith of NZGF. The 6,7-DHB may also be a concentrated liquid, produced by removal of water from the raw grapefruit juice or blend. The 6,7-DHB may also comprise a powder, formed by freeze-drying of the liquid material.

The 6,7-DHB may be a total extract, or an extract based on solubility in an extracting solvent, for example, a lipophilic or alcohol extract. The extract may be “enriched”, in that a first extract is further refined, using a second extraction, to concentrate desired constituents.

Quercetin is commercially available and may be obtained from a number of sources including Sigma Chemical Co., St. Louis, Mo.

Where the 6,7-DHB is a liquid, the quercetin may be dissolved in NZGF juice or other 6,7-DHB source to form the nutraceutical composition of the invention. Quercetin has low water solubility; therefore a nutraceutical composition of the invention may also include solvents to help quercetin dissolve. Examples of suitable solvents include alcohol and cyclodextrin, preferably β-cyclodextrin. Cyclodextrin can be used to increase the solubility of polyphenols in accordance with the teachings of US 20100204179.

Where the 6,7-DHB is a powder or solid, the nutraceutical composition of the invention may also be a powder or solid. Suitable powdered forms of 6,7-DHB include nettle root extracts, which are commercially available.

The composition may also comprise one or more nutraceutically acceptable excipients. Nutraceutically acceptable excipients are compounds, materials and compositions that are, inside the medical/nutritional fields, appropriate to use in touch with human and animal tissues without excessive toxicity, irritation, allergic response, or other proportionate problem or complication, with a reasonable risk-benefit ratio.

Nutraceutically acceptable excipients may include protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), flavorants, adsorbents, carriers, fillers, tableting agents, co-compounds, colorants, dispersing agents, emollients, surface-active agents, wetting agents, processing aids (solvents), flowing agents, taste-masking agents, weighting agents, jellifying agents, gel-forming agents, antioxidants and antimicrobials.

If the composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, Pa., USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives. Daily dosages are substantially the same as those above for food formulations, but for ease of administration, may be divided into smaller doses per administration unit, and multiple administration units (such as 1-4 capsules) may be taken daily.

In another embodiment the composition comprises one or more additional active agents.

Preferably, the composition is for oral administration.

It should be appreciated that while this invention preferably contemplates oral administration of the compositions of the invention, nothing herein should be construed to limit the mode of delivery. Both oral and systemic routes of delivery may be appropriate, particularly in combination-therapy regimes, ie, administration of a composition of the invention in combination with an additional active agent. Further, it should also be appreciated that any additional active agent need not be administered in the same manner, ie, one may be administered orally while another may be administered systemically.

Similarly, in the methods of the invention the 6,7-DHB and quercetin may be administered by different delivery routes. It therefore follows that while the compositions selected for the methods of the present invention are preferably administered concomitantly, the administration need not be co-instantaneously. It is preferred that they be administered such that their therapeutic effects are synchronized or overlap. Based upon ease of treatment, however, in a preferred embodiment, the selected compositions are administered separately, in individual unit dosage form.

Such unit dosage preparations may be adapted for oral administration as a tablet, capsule, liquid, powder, bolus, granule, elixir, dye, suspension, syrup, emulsion or the like.

Oral solid forms preferably have, in addition to active principle, a nutraceutically acceptable vehicle comprising one or more diluents, such as, lactose, dextrose, saccharose, cellulose, corn starch or potato starch; one or more lubricants, such as, silica, powder, stearic acid, magnesium or calcium stearate, or polyethylene glycols; one or more binding agents (agglutinants), such as, starches, mucilage, gelatin, methylcellulose, carboxymethylcellulose or polyvinyl pyrrolidone;

one or more disaggregating agents, such as, starch, alginic acid, alginates or sodium starch glycolates; effervescent mixtures; dyes; sugary agents; one or more humectant agents, such as, lecithin, polysorbate, laurylsulphate; and pharmacologically/nutraceutically inactive and non-toxic substances generally used in pharmaceutical/nutraceutical formulations and broadly known by pharmacotechnical workers.

They may likewise be prepared in unit dosage form, in an injectable form. They can be administered by infusion and intraperitoneal, subcutaneous, or intramuscular injection, all of them using known doses to those ordinarily skilled in pharmaceutical/nutraceutical art. They can be administered alone, but in general they are administered with a nutraceutically acceptable excipient selected from the basis of chosen administration route and from standard pharmaceutical/nutraceutical practice.

Dosage regimen for the composition of the present invention will vary according to the known factors, such as pharmacodynamic characteristics of the specific agents and administration route and modality, race, age, gender, health, medical condition, and receptor weight, symptom nature and extension; type of simultaneous treatment; treatment frequency; administration route, patient/user's hepatic and renal function, and aimed effect.

Suitable dosages of quercetin and/or 6,7-DHB will vary depending on the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prevention, data from cell culture assays or animal studies are used, wherein a suitable dose is within a range of circulating concentrations that include the ED₅₀ of the quercetin and/or 6,7-DHB with little or no toxicity. The dosage may vary within this range depending upon the dosage form employed and the route of administration utilized. A therapeutically/prophylactically effective dose can be estimated initially from cell culture assays.

A dose may be formulated in animal models to achieve a circulating plasma concentration range that includes the IC₅₀ (i.e., the concentration of the compound which achieves a half-maximal inhibition of symptoms) as determined in cell culture. Such information can be used to more accurately determine useful doses in humans. Levels in plasma maybe measured, for example, by high performance liquid chromatography.

In some embodiments, a method of the present invention comprises administering a prophylactically or therapeutically effective amount of quercetin.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of quercetin, rather the present disclosure encompasses any amount of quercetin sufficient to achieve the stated result in a subject.

As used herein, the term “prophylactically effective amount” shall be taken to mean a sufficient quantity of quercetin to prevent or inhibit or delay the onset of one or more detectable symptoms of a clinical condition. The skilled artisan will be aware that such an amount will vary depending on, for example, the particular subject and/or the type or severity or level of condition and/or predisposition (genetic or otherwise) to the condition. Accordingly, this term is not to be construed to limit the present disclosure to a specific quantity, e.g., weight or amount of querceting, rather the present disclosure encompasses any amount of quercetin sufficient to achieve the stated result in a subject.

For repeated administrations over several days or longer, depending on the severity of the disease or disorder to be treated, the treatment can be sustained until a desired suppression of symptoms is achieved.

In the case of a subject that is not adequately responding to treatment, multiple doses in a week may be administered. Alternatively, or in addition, increasing doses may be administered.

A subject may be retreated with quercetin, by being given more than one exposure or set of doses, such as at least about two exposures, for example, from about 2 to 60 exposures, and more particularly about 2 to 40 exposures, most particularly, about 2 to 20 exposures.

In one embodiment, any retreatment may be given when signs or symptoms of disease return.

Administration of quercetin and/or 6,7-DHB according to the methods of the present invention can be continuous or intermittent, depending, for example, on the recipient's physiological condition, whether the purpose of the administration is therapeutic or prophylactic, and other factors known to skilled practitioners. The administration of quercetin and/or 6,7-DHB may be essentially continuous over a preselected period of time or may be in a series of spaced doses, e.g., either during or after development of a condition.

Another aspect of the invention provides kits containing quercetin and 6,7-DHB. In one embodiment, the kit comprises (a) at least one container comprising quercetin and 6,7-DHB, optionally in a pharmaceutically/nutraceutically acceptable carrier or diluent; and (b) a package insert with instructions for treating or preventing a condition described herein. The quercetin and 6,7-DHB can be in the same or separate containers within the kit. The kit may also further comprise one or more other therapeutically active compounds or drugs.

The package insert is on or associated with the container. Suitable containers include, for example, bottles, vials, syringes, etc. The containers may be formed from a variety of materials such as glass or plastic. The container holds or contains the quercetin and/or 6,7-DHB and may have a sterile access port. The label or package insert indicates that the composition is used for treating a subject eligible for treatment, e.g., one having or predisposed to developing a condition described herein, with specific guidance regarding dosing amounts and intervals of the quercetin and/or 6,7-DHB and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprising a second medicament, and which kit further comprises instructions on the package insert for treating the subject with the second medicament, in an effective amount. The second medicament may be another compound that is currently being used or is in development for preventing or treating a condition described herein.

The compositions of the invention can be manufactured in a known way, for example, by means of mixture, granulation, tablet press, sugarcoating, or processes of film coating. Liquid nutraceutical forms for oral administration can be, for example, syrups, emulsions, or suspensions. Syrups can have a pharmaceutically or nutraceutically acceptable vehicle, such as, saccharose or saccharose with glycerin and/or manita (mannitol) and/or sorbitol. Suspensions and emulsion can have a pharmaceutically or nutraceutically acceptable vehicle, such as, a natural gum, agar, sodium alginate, pectin, methylcellulose, carboxymethylcellulose or polyvinilic alcohol. The forms intended for intramuscular injections can have, additionally to active principle, a pharmaceutically or nutraceutically acceptable vehicle, such as, sterile water, olive oil, ethyl oleate, propylene glycol and, an appropriate amount of lidocaine hydrochloride. In the present invention, oral forms are preferred.

The methods of the invention include preventing or treating a condition which would benefit from quercetin administration. Such conditions can include any disease, injury or other ailment which may benefit from quercetin's known biological activities (e.g., antioxidant and/or ionophore properties). Therapeutic uses of quercetin will be known by those skilled in the art and include uses described in Salehi et al., 2020. For example, conditions that would benefit from administration of quercetin include cancers, inflammation, oxidative stress, ageing, hair loss, autoimmune diseases, allergies, cardiovascular diseases, hypertension, hormonal conditions, neurodegenerative diseases, dysbioses, and infections.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The present application claims priority from AU 2020901205 filed 16 Apr. 2020 and NZ 762991 filed 26 Mar. 2020, the entire contents of which are incorporated herein by reference.

### 6. EXAMPLES

### Example 1: Human Trial Showing Increase in Bioavailability of Quercetin when Administered with Grapefruit Extract

The concentration of quercetin in the blood of a human subject was measured over four hours following ingestion of:
(a) 80 mg quercetin and 200 ml filtered water,
(b) 80 mg quercetin and 200 ml grapefruit extract.

The grapefruit extract consisted of grapefruit juice that was squeezed from the grapefruit with a juicer. The collected juice was freeze-dried and redissolved as a concentrate (4 times) using water for solubilising.

The total amount of 6,7-DHB administered in the grapefruit extract in (b) above was estimated to be about 5.9 mg.

In both experiments, the quercetin was administered as a powder washed down with filtered water.

For the quercetin and grapefruit extract test, the grapefruit extract was taken two hours before the quercetin.

Serum samples were taken from the subject at 15 minute intervals. The samples were extracted into ethyl acetate and the concentration of quercetin determined using HPLC with UV detection at 315 nm. Carbamazepine was used as an internal standard.

A linear relationship for the standard curve was achieved over the range of quercetin standards of 2-150 ng/mL (using weighted linear retgression 1/x²).

The lower limit of quantification (LLOQ) for the method was determined to be 2 ng/mL (using FDA guidelines for bio-analytical procedures) and the limit of detection was estimated to be 1 ng/mL.

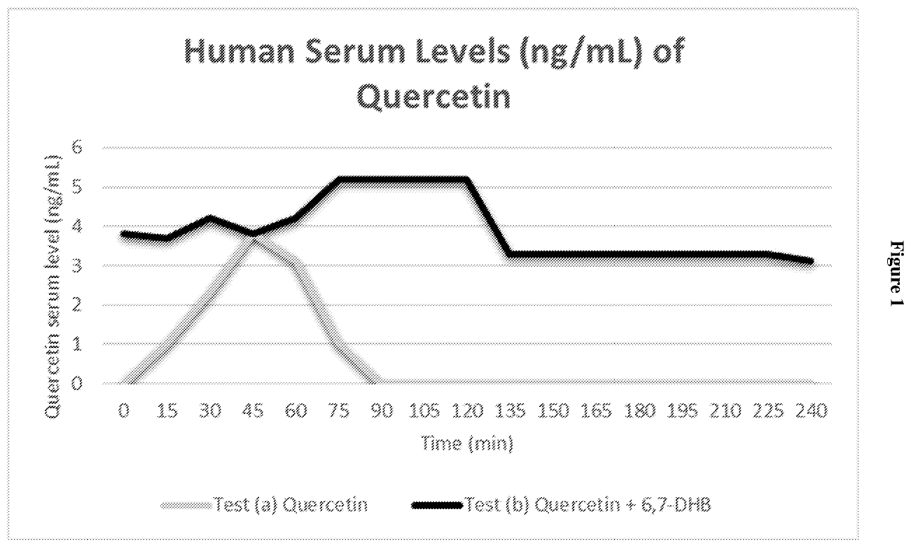

The results are shown in Table 1 and FIG. 1.

TABLE 1
Serum levels of quercetin following ingestion of (a) quercetin
and (b) grapefruit extract followed by quercetin

| Time | Test (a) | Test (b) |
|---|---|---|
| (min) | (ng/mL) | (ng/mL) |
| 0 | 0 | 3.8 |
| 15 | 1 | 3.7 |
| 30 | 2.3 | 4.2 |
| 45 | 3.8 | 3.8 |
| 60 | 3.1 | 4.2 |
| 75 | 1 | 5.2 |
| 90 | 0 | 5.2 |
| 105 | 0 | 5.2 |
| 120 | 0 | 5.2 |
| 135 | 0 | 3.3 |
| 150 | 0 | 3.3 |
| 165 | 0 | 3.3 |
| 180 | 0 | 3.3 |
| 195 | 0 | 3.3 |
| 210 | 0 | 3.3 |
| 225 | 0 | 3.3 |
| 240 | 0 | 3.1 |

When quercetin was taken following administration of grapefruit extract, quercetin levels were much higher and remained higher throughout the duration of the study, indicating increased bioavailability. Interestingly, the zero point concentration of quercetin in the serum was quite high.

### Example 2: Bioavailability of Quercetin in Rats Following Administration of American Ruby Red Grapefruit Extract and Grapefruit Extract

Rats were administered:
(a) Quercetin
(b) American Ruby Red grapefruit extract
(c) Quercetin and American Ruby Red grapefruit extract
(d) grapefruit extract
(e) grapefruit extract and quercetin

The methodology for the administration of quercetin to rats was based on a number of publications (Harper, Cook et al, 2009; Csiszar, Labinskyy et al, 2009; Sengottuvelan, Deeptha, Nalini, 2009).

The administration of the grapefruit juice to rats was based on a number of publications (de Castro, Mertens-Talcott et al, 2008; Gorinstein, Leontowicz et al, 2005; Saadeddin, Torres-Molina et al, 2004)

(i) Animals

Each group was studied independently in terms of scheduling.

5 groups of 8 male Sprague-Dawley rats, aged approximately 14 weeks, weighing 350-400 gm were used in the study. The rats were bred at Hercus-Taeri Resource Unity. All of the rats were healthy and had no medication history.

The rats were fed an unsupplemented standard rodent diet and filtered tap water.

The testing room and rat boxes were cleaned prior to beginning the study. The room was also disinfected before commencement of the study. The temperature of the room during the test was maintained between 18° C. and 22° C. Illumination involved a 12 h light/dark cycle.

(ii) Preparation of Test Materials

1. The quercetin was dissolved in distilled water at 12.0 mg/ml immediately prior to administration.

2. American Ruby Red grapefruit was cut in half and juice obtained by manual squeezing.

The grapefruit extract used was a commercially obtained grapefruit juice.

3. A known volume of each of the grapefruit juices was lyophilized. Once dry, the residue was resuspended in distilled water to 25% of its original volume. This reduced the volume to be gavaged to each rat in the relevant groups to make the quantities of active agents administered as high as practical.

4. The quercetin/grapefruit juice formulations were prepared by dissolving the quercetin in the concentrated juices at 12.0 mg/ml.

(iii) Groups of Rats

There were 5 groups of rats (8 males per group) as follows:

GROUP 1. Each animal in this group received quercetin at 15 mg per kg body weight administered by oral gavage.

GROUP 2. Each animal in this group received the concentrated New Zealand grapefruit juice (American Ruby Red) at 2.5 ml per kg body weight administered by oral gavage.

GROUP 3. Each animal in this group received quercetin dissolved in the concentrated New

Zealand grapefruit juice (American Ruby Red) administered by oral gavage. The juice was administered so as to provide 15 mg quercetin per kg body weight.

GROUP 4. Each animal in this group received the concentrated grapefruit juice (prepared by Cedenco Ltd) at 2.5 ml per kg body weight administered by oral gavage.

GROUP 5. Each animal in this group received quercetin dissolved in the concentrated grapefruit juice (prepared by Cedenco Ltd) at 2.5 ml per kg body weight administered by oral gavage. The quercetin was dissolved so that it was administered at 15 mg per kg body weight.

(iv) Experimental Procedures

Each group of rats was studied independently. For each group:

1. A 0.5 ml time zero blood sample was taken from each animal from the lateral saphenous vein. The animals were anaesthetized using Halothane in oxygen (3.5% for knockdown, 2.5 to 3.0% for maintenance). The vein was cannulated. The zero-time blood sample was taken from each group of rats the day before the animals were gavaged. Following the collection of the blood sample, a 0.5 ml aliquot of sterile normal saline was given by intraperitoneal (IP) injection.

2. The test sample was prepared freshly immediately prior to the gavaging of each group of rats. Each animal was weighed and the volume of the test sample to be administered calculated.

3. Each rat was gavaged using proprietary gavaging needles (Popper). Once gavaging was complete the animals were left for 10 minutes before being anaesthetized using Halothane in oxygen (3.5% for knockdown, 2.5 to 3.0% for maintenance).

4. Blood samples (0.50 ml) were taken at TO (as described in step 1 above), 30, 60 and 120 minutes from the anesthetized rat using a 1 ml graduated syringe. After each collection an equal volume of sterile saline was administered to each animal via an IP injection. The blood samples were immediately transferred to serum separating microtainer tubes (Becton Dickinson Cat. No 365956) and left to clot for 30 minutes at room temperature. The tubes were centrifuged at 3000 rpm for 10 minutes in a Beckman J6 centrifuge with a swing-out rotor. The sera were pipetted off and transferred into Eppendorf tubes/cryovials which were stored at −80° C.

5. After the T120 minute blood sample had been taken each rat was euthanized by cervical dislocation.

6. As the blood sampling times were relatively frequent, each group's treatment was spread out over two days and hence the time to treat all five groups was 10 days (4 animals on each day).

(v) Analyses

The sera samples were sent to the Institute of Environmental Science and Research (ESR) (Kenepuru, Wellington, New Zealand) for quantitation of the concentration of quercetin in each. The detection limit for quercetin in serum was 5 ng/ml.

(vi) Results and Discussion

i) Summary

For the rats that were administered quercetin only, none was detected in any of the animals even at 30 minutes after administration.

For the rats that were administered the American Ruby Red grapefruit, there was no quercetin detected in any of the animals even at 30 minutes after administration.

Of the eight rats administered quercetin and American Ruby Red grapefruit, two showed quercetin in the serum still being present after 2 hours. Another two showed quercetin in the serum at 30 minutes, but it was not detected after that.

For the rats that were administered the grapefruit extract, there was no quercetin detected in seven of the eight animals even at 30 minutes after administration. One rat showed quercetin in the serum 30 minutes after administration which was still detectable after 1 hour.

Of the eight rats administered quercetin and grapefruit extract, six showed quercetin in the serum after 30 minutes. Five of these six still had significant levels of quercetin in the serum one hour after administration. Two hours after the administration four of the animals still displayed measurable levels of quercetin.

ii) Results

The concentration of quercetin in the serum from individual rats is summarised in the Table below. Data from three of the rats (C2, C8 and E3) was discarded as being tainted. Blood serum of these animals indicated high levels of quercetin at each time point including TO. As the TO blood sample was taken the day before the rates were gavaged, clearly the data was unreliable.

TABLE 2
Quercetin alone

| Time | A1 | A2 | A3 | A4 | A5 | A6 | A7 | A8 |
|---|---|---|---|---|---|---|---|---|
| 0 min | 0 | 0 | 0 | 0 | 0 | 0 | 0 | 0 |
| 30 min | 0 | 0 | 0 | 0 | 0 | 0 | 0 | 0 |
| 60 min | 0 | 0 | 0 | 0 | 0 | 0 | 0 | 2 |
| 90 min | 0 | 0 | 0 | 0 | 0 | 0 | 0 | 2 |
| 120 min | 0 | 0 | 0 | 0 | 0 | 0 | 0 | 2 |

TABLE 3
American Ruby Red grapefruit alone

| Time | B1 | B2 | B3 | B4 | B5 | B6 | B7 | B8 |
|---|---|---|---|---|---|---|---|---|
| 0 min | 0 | 0 | 0 | 0 | 0 | 0 | 0 | 0 |
| 30 min | 0 | 0 | 0 | 0 | 0 | 0 | 0 | 0 |
| 60 min | 0 | 0 | 0 | 0 | 2 | 0 | 0 | 0 |
| 90 min | 0 | 0 | 0 | 0 | 1 | 0 | 0 | 0 |
| 120 min | 0 | 0 | 0 | 0 | 0 | 0 | 0 | 0 |

TABLE 4
American Ruby Red grapefruit + Quercetin

| Time | C1 | C2 | C3 | C4 | C5 | C6 | C7 | C8 |
|---|---|---|---|---|---|---|---|---|
| 0 min | 0 | Removed | 0 | 0 | 0 | 0 | 0 | Removed |
| 30 min | 0 | Removed | 0 | 0 | 12.3 | 0 | 11.4 | Removed |
| 60 min | 0 | Removed | 0 | 0 | 0 | 0 | 0 | Removed |
| 90 min | 0 | Removed | 0 | 0 | 0 | 0 | 0 | Removed |
| 120 min | 0 | Removed | 0 | 0 | 0 | 0 | 0 | Removed |

TABLE 5
grapefruit extract alone

| Time | D1 | D2 | D3 | D4 | D5 | D6 | D7 | D8 |
|---|---|---|---|---|---|---|---|---|
| 0 min | 0 | 0 | 0 | 0 | 0 | 0 | 0 | 0 |
| 30 min | 0 | 19.5 | 0 | 0 | 0 | 0 | 2 | 0 |
| 60 min | 0 | 9.4 | 0 | 0 | 0 | 0 | 2 | 2 |
| 90 min | 0 | 4.7 | 0 | 0 | 0 | 0 | 2 | 1 |
| 120 min | 0 | 0 | 0 | 0 | 0 | 0 | 2 | 0 |

TABLE 6
grapefruit extract + Quercetin

| Time | E1 | E2 | E3 | E4 | E5 | E6 | E7 | E8 |
|---|---|---|---|---|---|---|---|---|
| 0 min | 0 | 0 | Removed | 0 | 0 | 0 | 0 | 0 |
| 30 min | 25.2 | 13.9 | Removed | 9.4 | 0 | 0 | 26.3 | 35.6 |
| 60 min | 0 | 15.6 | Removed | 9.3 | 0 | 0 | 25.1 | 46.9 |
| 90 min | 1 | 8.8 | Removed | 9.35 | 0 | 0 | 24.35 | 32.25 |
| 120 min | 2 | 2 | Removed | 9.4 | 0 | 0 | 23.6 | 17.6 |

Removed=Animals that were removed from the study, prior to completion of the study.

### Example 3: An Exemplary Composition

An exemplary composition (in capsule form) is provided below in Table 7. Preferred quantities of each ingredient are listed with preferred ranges in parentheses.

TABLE 7
exemplary composition for enhancing bioavailability of quercetin

| | Preferred Quantity | |
|---|---|---|
| Ingredient | mg/capsule | |
| Vitamin D3 | 5 | (1 to 20) |
| Quercetin, Anhydrous 95% granulation | 526 | (100 to 1500) |
| Ginger extract 5% Gingerols | 40 | (10 to 80) |
| Nettle Root HPLC extracted 6,7-DHB (45%) | 15* | (2 to 50) |
| Piper Nigrum fruit Extract | 5 | (1 to 20) |
| Vitamin C | 55 | (10 to 100) |
| Zinc gluconate | 22.9\hat{ } | (5 to 50) |
| Microcrystalline cellulose (MCC) | 83 | (20 to 200) |
| Silica Colloidal Anydrous | 7 | (1 to 20) |
| Magnesium Stearate | 5 | (1 to 20) |

*15 mg of 45% 6,7-DHB equates to 6.75 mg of pure 6,7-DHB
\hat{ }22.9 mg of zinc gluconate equates to about 3.3 mg of zinc.

Rationale for Ingredients

Vitamin D3: Helps to maintain serum Vit D levels in a healthy range. Population studies suggest that insufficient serum Vit D has been associated with increased respiratory infections.

Quercetin: quercetin acts as an antioxidant and a natural zinc ionophore which can transport zinc across the cellular membrane into the cell, independently of covalent ion transporter molecules that are native to the cell membrane. However, due to its low bioavailability and poor absorption characteristics the concentration of quercetin required in conventional supplements is approximately 5 g per dosage. The above exemplary dosage comprises only approximately 0.5 g.

Ginger root extract: This compound has a number of known health benefits and further improves the absorption and subsequent bioavailability of quercetin.

Nettle Root HPLC-extracted 6,7-DHB (45%): The inventors have shown that 6,7-DHB improves the absorption and subsequent bioavailability of quercetin, thereby reducing the requirement of quercetin from 5 g to approximately 0.5 g to 1 g. The nettle root used has a high potency due to the extraction process utilised.

Piper nigrum fruit extract: This compound is reported to have anti-oxidant, anti-bacterial, anti-tumor, anti-mutagenic, anti-diabetic, and anti-inflammatory properties. It also further improves the absorption and subsequent bioavailability of quercetin.

Vitamin C Known antioxidant. This compound further improves the absorption and subsequent bioavailability of quercetin.

Zinc: The effectiveness of quercetin (a zinc ionophore) will be reduced if the subject is either deficient in zinc or has a serum insufficiency. Added zinc ensures optimum serum zinc for quercetin to work most efficiently as an ionophore.

Other Optional Ingredients

RiboCeine (D-ribose-L-cysteine): 125 mg (75 mg to 250 mg). If this ingredient is included then it may be necessary to split the above exemplary composition into two separate capsules.

Dosage Regime

Optimal dosage for a human subject is one or two capsules defined in Table 7 above, per day.

### REFERENCES

Kaşikci M. B, Bağdathoğlu N. Bioavailability of Quercetin. Curr Res Nutr Food Sci 2016;4(Special Issue Confernce October 2016). doi: http://dx.doi.org/10.12944/CRNFSJ.4.Special-Issue-October.20
Williams R J, Spencer J P, Rice-Evans C (April 2004). “Flavonoids: antioxidants or signalling molecules?”. Free Radical Biology & Medicine. 36 (7): 838-49.
Russo G L, Russo M, Spagnuolo C, Tedesco I, Bilotto S, Iannitti R, Palumbo R (2014). “Quercetin: a pleiotropic kinase inhibitor against cancer”. Cancer Treatment and Research. 159: 185-205.
Graefe E U, Derendorf H, Veit M (1999). “Pharmacokinetics and bioavailability of the flavonol quercetin in humans”. International Journal of Clinical Pharmacology and Therapeutics. 37 (5): 219-33.
Lown Kans., Bailey D G, Fontana R J, Janardan S K, Adair C H, Fortlage, La., Brown M B, Guo, W, Watkins, P B (1997). Grapefruit Juice Increases Felodipine Oral Availability in Humans by Decreasing Intestinal C Y P3A Protein Expression. J Clin Invest 99: 2545-2553
Harper, C E Cook, L M, Patel, B B, Wang, J, Eltoum, I A, Arabshahi, A, Shirai, T, Lamartiniere, C A (2009). Genistein and Quercetin, Alone and in Combination, Suppress Prostate Cancer in SV-40 Tag Rats, The Prostate 69: 1668-1682
Csiszar, A, Labinskyy, N, Olson, S, Pinto, J T, Gupte, S, W u, J M, H u, F, Ballabh, P, Podlutsky, A, Losonczy, G, de Cabo, R, Matthew, R, Wolin, Miss., Ungvari, Z (2009). Quercetin Prevents Monocrotaline-Induced Pulmonary Hypertension in Rats. Hypertens 54: 668-675
Sengottuvelan, M, Deeptha, Nalini, N (2009). Quercetin Ameliorates DNA Damagge, Prooxidant and Antioxidant Imbalance in 1,2-Dimethylhybrazine Induced Rat Colon Carcinogenesis. Chem—Biol Interactions 181: 193-201
de Castro, W V, Mertens-Talcott, S, Derendorf, H, Butterweck, V (2008). Effect of Grapefruit Juice, Naringenin, and Bergamottin on the Intestinal Carrier-Mediated Transport of Talinolol in Rats. J Agric Food Chem 56: 4840-4845
Gorinstein, S, Leontowicz, H, Leontowicz, M, Krzeminski, R, Gralak, M, Delgado-Licon, E, Ayala, ALM, Katrich, E, Trakhtenberg, S (2005). Changes in Plasma Lipid and Antioxidant Activity in Rats as a Result of Naringin and Red Grapefruit Supplementation. J Agric Food Chem 53: 3223-3228
Saadeddin, A, Torres-Molina, F, Cárcel-Trullols, J, Araico, A, Peris, J E (2004). Effect of Cytochrome P-450 Inhibitors (Diethyl Dithiocarbamate, Ketoconazole and Grapefruit Juice) on the Pharmacokinetics of All-trans-retinoic acid. II Farmaco, 59: 697-702
Bahare Salehi, Laura Machin, Lianet Monzote, Javad Sharifi-Rad, Shahira M. Ezzat, Mohamed A. Salem, Rana M. Merghany, Nihal M. El Mandy, Ceyda Sibel Kiliç, Oksana Sytar, Mehdi Sharifi-Rad, Farukh Sharopov, Natalia Martins, Miguel Martorell, and William C. Cho (2020) ACS Omega 5:11849-11872
Read S A, Obeid S, Ahlenstiel C, Ahlenstiel G. (2019) The Role of Zinc in Antiviral Immunity. Adv Nutr. 10(4):696-710

1. A composition comprising quercetin and 6,7-dihydroxybergamottin (6,7-DHB), wherein 6,7-DHB is present in an amount sufficient to enhance the bioavailability of quercetin.

2. The composition of claim 1, wherein the quercetin is present in an amount in the range of about 100 mg to about 1,500 mg, or about 250 mg to about 750 mg, or about 400 mg to about 600 mg.

3. The composition of claim 1, wherein the 6,7-DHB is present in an amount in the range of about 1 mg to about 50 mg, or about 2 mg to about 20 mg, or about 4 mg to about 10 mg.

4. The composition of claim 1, wherein the 6,7-DHB is present in the form of a nettle root extract or a grapefruit extract.

5. The composition of claim 1, which comprises one or more or all of the following:
(a) zinc;
(b) vitamin D3;
(c) vitamin C;
(d) Piper nigrum fruit extract; and
(e) ginger root extract.

6. The composition of claim 1, which comprises one or more nutraceutically acceptable excipients.

7. The composition of claim 6, wherein the nutraceutically acceptable excipients include one or two or all of microcrystalline cellulose, colloidal silica, and magnesium stearate.

8. The composition of claim 1, which comprises D-ribose-L-cysteine.

9. The composition of claim 8, wherein the D-ribose-L-cysteine is present in an amount in the range of about 75 mg to about 250 mg.

10. The composition of claim 1, which is a dry composition.

11. A nutraceutical composition comprising quercetin and a source of 6′,7′-DHB, and optionally nutraceutically acceptable excipients.

12. The composition of claim 1 consisting essentially of quercetin and 6,7-DHB.

13. A method of preventing or treating a condition in a subject, which condition would benefit from administration of quercetin, the method comprising administering the composition of claim 1 to the subject.

14. A method of preventing or treating a condition in a subject, which condition would benefit from administration of quercetin; preventing or treating cancer, inflammation, cardiovascular disease, hypertension, polycystic ovary syndrome, a neurodegenerative disease, or an infection in a subject increasing the bioavailability of quercetin in a subject; enhancing cellular zinc uptake in a subject; or enhancing probiotic activity in a subject; the method comprising administering to the subject 6,7-DHB, simultaneously or sequentially with quercetin.

15. The method of claim 13, wherein the condition is a cancer, inflammation, cardiovascular disease, hypertension, polycystic ovary syndrome, a neurodegenerative disease, or an infection.

16-20. (canceled)

21. The method of claim 14, wherein the 6,7-DHB and quercetin are administered in the same composition or in separate compositions.

22. (canceled)

23. (canceled)

24. The method of claim 14, wherein the 6,7-DHB is administered to the subject prior to quercetin.

25. The method of claim 24, wherein the 6,7-DHB is administered about 0.5 to about 12 hours prior to quercetin.

26. The method of claim 14, wherein quercetin is administered in an amount in the range of about 100 mg to about 1,500 mg, or about 250 mg to about 750 mg, or about 400 mg to about 600 mg per dose.

27. The method of claim 14, wherein 6,7-DHB is administered in an amount in the range of about 1 mg to about 50 mg, or about 2 mg to about 20 mg, or about 4 mg to about 10 mg per dose.

28. The method of claim 14, wherein one or two doses of quercetin are administered per day and/or one or two doses of 6,7-DHB are administered per day.

29. The method of claim 14, further comprising administering zinc to the subject.

30. The method of claim 29, wherein zinc is administered in an amount in the range of about 1 mg to about 20 mg per dose.

31. (canceled)
